(19) United States
(12) Patent Application Publication
(10) Pub. No.: US 2024/0123248 A1
(43) Pub. Date: Apr. 18, 2024

(54) TRANSCRANIAL MAGNETIC STIMULATION SYSTEM AND METHOD

(71) Applicant: Ampa Inc., San Diego, CA (US)

(72) Inventors: Donald A. VAUGHN, San Diego, CA (US); Jonathan A.S. DOWNAR, Carrying Place (CA)

(21) Appl. No.: 18/212,055

(22) Filed: Jun. 20, 2023

Related U.S. Application Data
(63) Continuation-in-part of application No. 17/964,686, filed on Oct. 12, 2022, now Pat. No. 11,730,969.

(51) Int. Cl. A61N 2/00; A61B 5/00; A61N 2/02

(52) U.S. Cl. CPC A61N 2/006; A61B 5/0036; A61B 5/0042; A61B 5/4064; A61B 5/442; A61B 5/446; A61B 5/4504; A61B 5/6803; A61B 5/6842; A61N 2/02

(57) ABSTRACT

A transcranial magnetic stimulation system includes a magnetic field generator configured to generate a magnetic field to be applied to a patient's head, the magnetic field generator comprising one or more magnetic induction coils, a housing for the coils, and a Phase Change Material (PCM) in contact with the coils contained in the housing. One or more imaging devices configured to permit direct visualization of the coils on the patient's head are embedded in the housing. The one or more imaging device(s) may include one or more cameras, preferably one or more visible light imaging cameras, one or more ultraviolet light imaging cameras, or one or more infrared imaging cameras.

### CROSS REFERENCE TO RELATED APPLICATION

This application is a Continuation-in-Part (CIP) of U.S. Application Ser. No. 17/964,686, filed Oct. 12, 2022, the contents of which are incorporated herein by reference.

### BACKGROUND OF THE DISCLOSURE

The present disclosure relates to a system for transcranial magnetic stimulation (TMS), i.e., methods and apparatus for positioning a transcranial magnetic stimulation device properly on the head of patients so as to deliver magnetic stimulation to a specific brain region. The disclosure has particular applicability to systems and methods for applying magnetic stimulation to target brain regions of a patient for treating depression and will be described in connection with such utility, although other utilities are FDA-approved including treatment for obsessive compulsive disorder (OCD), depression with comorbid anxiety, and nicotine addiction, and still others show promising early results including TMS for treatment of bipolar disorder, post-traumatic stress disorder (PTSD), eating disorders, personality disorders, alcohol and other substance use disorders, and autism, as well as neurological illnesses including Alzheimer's Disease and other dementias, migraine headaches, movement disorders such as Parkinson's Disease, tinnitus, and chronic pain.

For each of these disorders, a distinct set of brain regions is known to be functioning abnormally, and one or more of these regions must be located and accurately targeted during stimulation for successful treatment. In order to reliably stimulate a desired brain region, the TMS coil needs to be consistently and accurately placed at a target scalp location overlying that brain region and must remain at that site throughout the entire stimulation session. Stimulation of off-target brain regions may reduce or eliminate the efficacy of the treatment, and in the worst-case scenario may lead to worsening of symptoms, excessive pain during treatment, or rarely, a serious adverse event such as a seizure.

The process of positioning the TMS coil on the head, and maintaining it in place during treatment, is known as ‘neuronavigation.’ In current clinical practice, the most common approach to neuronavigation is to place a fabric cap on a patient's head, perform measurements of the head and scalp, and use these measurements to define a coordinate system (e.g., the ‘10-20 international encephalography system’) which provides markers on the patient's head. A TMS operator would then use these marks to specify the target site on the cap, place the TMS coil over the marked target site, and then trace a (partial) outline of the coil on the cap for use in maintaining consistent coil orientation during treatment sessions, and from session to session. However, this method may be imprecise, and does not provide direct visual confirmation that the center of the coil is (1) directly over the target location, or (2) actually in physical contact with (i.e., touching) the patient's head.

In addition, patients may move during treatment and/or navigational aids may slip out of the desired position. If this occurs, the TMS technician must pause treatment, readjust the position of the coil, and then resume stimulation. At worst, a patient can move his or her head in such a way that the TMS coil moves but is not observably positioned off target such that the session continues, with potential adverse consequences as identified above. Present methods lack a direct visual or other record confirming that (1) the TMS coil is optimally located over the desired target area, (2) the TMS coil remains over the desired target area throughout the stimulation session, and that (3) the TMS coil remains in physical contact with the surface of the patient's head during the entire session.

A more complex, less commonly used approach to neuronavigation (FIG. 1) involves a computerized frameless stereotaxic positioning system comprised of: (1) a set of optical position markers such as small reflective beads 70 attached in a specific 3-dimensional configuration to the coil as well as to a tracker on the patient's head, (2) a stereo camera 71 that visualizes and localizes the markers in 3-dimensional space, and (3) computer software 72 which uses 3-dimensional marker position information from the camera to impute the relative positions and orientations of the patient's head and the coil, and then provides a visualization of these positions on a screen viewed by the operator as a neuronavigation guide before and during treatment. Such a system usually, but not always, also includes (4) an MRI or other 3-dimensional image of the patient's head and brain, which the software aligns with the 3-dimensional imputed head position, so that the operator may visualize the brain region at the focus of the coil in real-time during coil positioning and treatment.

As examples, JP 2003-180649A and JP 2004-000636A disclose techniques for TMS coil neuronavigation using, for example, an optical tracking system employing infrared reflectors, as described above. This technology is commercially available and is also used in clinical settings such as neurosurgical procedures requiring neuronavigation.

JP 2006-320425A discloses another apparatus for positioning a TMS coil against the patient's head by using a multi jointed robot. This approach likewise has several major disadvantages, including the necessity of an MRI scan for every patient, the excessive additional expense and complexity of the apparatus itself, and the need for an operator to undergo an extended training period of several additional weeks to achieve proficiency in accurate use of the system. Further, the system can fail if: (1) the specified target is mistaken, (2) the markers on the coil are incorrectly calibrated, (3) following calibration, the markers on the patient's head move out of position during the session, (4) the operator is insufficiently skilled, or if (5) the coil is not quite in contact with the scalp despite appearing to be so on the neuronavigation system.

The complexity of this external tracking approach with MRI-guidance also greatly reduces the variety, and hence numerosity, of locations where patients may receive TMS treatment. This system is typically confined to a hospital setting because it requires high-field MRIs, as well as significant computing resources, specialized analysts to process the images, and technicians trained to competently operate the cumbersome neuronavigation suite. As a result, this approach is rarely used in the most accessible health care settings such as primary care clinics, mental health centers, assisted living facilities, outpatient specialty clinics, or workplace health centers. Instead, a patient seeking MRI-guided neuronavigated TMS is generally obliged to repeatedly travel to an academic or tertiary health care setting, raising additional barriers of cost and convenience and curtailing the accessibility of TMS treatment for those who need it.

The foregoing discussion of the prior art derives in part from U.S. Pat. No. 10,004,915 (the '915 patent) wherein there is described a TMS system comprising a TMS alignment system comprising a means for generating magnetic field, the magnetic field generating means having a coil for generating a variable magnetic field to be applied to a certain part of patient's head and a holder for holding the coil; and a camera means for recognizing a predetermined reference marking made on a specific portion of the ear of the patient, (e.g., the tragus); the magnetic field generating means and the recognizing means being designed so that an alignment of the recognizing means with the marking causes the coil to be set in a proper posture with respect to the certain part of the patient's head.

According to the '915 patent, with the aforesaid arrangement, the magnetic field generating means can be positioned with respect to the reference marking of a specific portion the patient's ear, allowing the user of the TMS system to position the magnetic field generating means without skill which is needed for conventional systems.

The recognition means of the '915 patent includes at least one imaging device, i.e., cameras carried on external arms extending from an apparatus. Alignment includes aligning an optical axis of the imaging device with the marking. This allows the coil to be positioned in the proper posture with respect to the specific part of the patient.

Preferably, the TMS system of the '915 patent further comprises an optical device capable of emitting a directional beam, the optical device being provided adjacent the imaging device, wherein the alignment includes aligning an intersection of the optical axis of the optical device with the marking. This allows the TMS coil to be positioned in the proper position with respect to the specific part of the patient.

In another embodiment of the '915 patent, the TMS system further comprises a moving mechanism for moving the coil holder on and along a surface of the patient's head; and a controlling means for controlling the moving mechanism in accordance with an output from the recognition means to automatically position the holder with respect against the marking.

As noted earlier, a problem with the TMS alignment system proposed in the '915 patent is that the system requires marking directly on the patient, the system is bulky, and the arms required for holding the cameras or imaging devices are bulky and themselves prone to bending and/or misalignment. The alignment markings on the patient can also be obscured by the patient's hair. The additional components also bear the risk of inaccurately imputing the coil's actual position, as explained above. Lastly, the additional components reduce the overall accessibility of TMS treatment, by requiring technicians to undergo extensive additional training in order to operate the neuronavigation apparatus correctly, and by limiting treatment location to areas where a stationary multi-ton MRI scanner happens to reside.

### SUMMARY OF THE INVENTION

The present disclosure is based on the premise that major sources of potential error and uncertainty of treatment can be removed from the process of TMS coil neuronavigation if: (1) the target site on the head can continuously be precisely visualized under the center of the coil, and (2) a contact sensor can directly indicate whether the center of the TMS coil is in contact with the scalp during the entire stimulation session. In one embodiment, we provide an optical scalp-landmarking approach which allows for much higher consistency in positioning a TMS coil over a given site on the scalp from session to session, as well as providing a direct visual record (as opposed to an imputed calculation) of whether the coil was properly positioned and maintained in this position and in contact with the scalp consistently throughout each session of stimulation. Said another way, rather than using externally placed sensors and markers on the head to infer the coil location from an external perspective (as in FIG. 1), our approach uses sensors placed on the coil itself to, in essence, provide the coil's perspective. Moreover, since our novel approach allows the technician and supervising physician to directly visualize the target site while placing the coil, the time required to train a new technician to proficiency is greatly reduced, rather than extended. Moreover, a verifiable record of placement accuracy may be generated during each treatment session. Finally, our approach does not necessitate the use of costly and cumbersome additional components such as stereo cameras, MRI machines and intensive processing software, or reflector markers requiring calibration prior to treatment. This reduction in cost and complexity, as well as the marked acceleration of an operator's learning curve to proficiency, facilitates more widespread access to neuronavigated TMS treatment in a broader range of settings—outside the more limited number of specialized centers which host large, expensive apparatus, requiring extensive personnel.

In accordance with one embodiment, we incorporate one or more imaging devices into a TMS coil configured to permit direct visualization of the placement of the center of the TMS coil on the head. In one embodiment, we incorporate a single camera directly in the center of the TMS coil configured to permit direct visualization of the area under a TMS coil. In another embodiment of the disclosure, we incorporate two or more cameras placed off-center and/or on the sides of the TMS coil. The camera(s) may comprise visual light imaging capabilities, ultraviolet light imaging capabilities or infrared light imaging capabilities.

In another embodiment, we also incorporate into the TMS coil one or more contact sensors configured to detect whether the patient's head is in contact with the coil before, during, and until the treatment session concludes. The contact sensors may comprise force-sensitive resistors, capacitive touch sensors, ultrasonic position/touch sensors, and/or thermal/infrared sensors.

In another embodiment, we also incorporate one or more imaging devices external to the coil, configured to allow for simultaneous visualization of the patient's head (and any associated markings) as well as the coil, as an independent measure of their relative positions. These additional coil-external cameras may comprise one or more cameras, LIDAR detectors, and/or ultrasonic detectors.

In yet another embodiment, we provide a specialized treatment cap having indicia with various markings including grid markings, text and/or color markings corresponding to specific anatomical locations on the head of the patient.

In yet another embodiment, rather than employing the current standard of care of placing the treatment cap a few centimeters above the eyebrows, measuring the distance from nasion to cap brim, and then for every subsequent session, trying to place the cap back exactly that same distance, remeasuring each time, we provide a cap geometry where the brim comes to a point on the midline. This point can be immediately visually confirmed to be correctly placed or not without the need for measuring tape. Additionally, we place indicia on the cap that denotes where the cap should be with respect to the tragus (the point flap of skin on the ear), as an additional marker to ensure reliable fit of the cap on the head.

With both the point front, and tragus markers on both sides, these three markers can be used not only for basic visual confirmation, but we can employ AI algorithm to ensure proper cap positioning, by using a smartphone camera and slowly wave it around the patient from left to front to right side to ensure the cap is properly positioned.

In another embodiment, the TMS system is configured to record and optionally transmit, in real time, a video of the TMS coil placement during treatment. Also, in yet another embodiment, we include one or more accelerometers in the TMS coil configured to provide a supplementary record of the orientation of the TMS coil throughout treatment, so that the provider can detect any subtle drift or deviation of the coil during treatment and make adjustments to the TMS coil orientation accordingly.

In another embodiment we provide a transcranial magnetic stimulation system comprising: a TMS system configured to generate a magnetic field to be applied to a patient's brain region, the TMS system comprising a transcranial magnetic stimulation (TMS) pulse generator as well as an inductor coil; and one or more imaging devices incorporated into the coil and configured to permit direct visualization of the TMS coil on the patient's head. The imaging device may comprise one or more cameras, preferably one or more visible light imaging cameras, one or more ultraviolet light imaging cameras, or one or more infrared imaging cameras.

The transcranial magnetic stimulation system may further comprise one or more accelerometers configured to sense orientation placement and/or changes in orientation of the TMS coil.

The transcranial magnetic stimulation system also may further comprise a memory device configured to create a video record of TMS coil placement during treatment.

We also provide a transcranial magnetic stimulation neuronavigation kit, comprising the transcranial magnetic stimulation system as above described, and patient head cap having grid markings, text and/or color markings configured to overlie anatomical locations on the head of the patient. The patient head cap may include markings configured to overlie target areas of the head of the patient, and/or markings configured to permit continuous measurement of the position and orientation of the cap relative to the patient's head before, during, and after treatment.

A feature and advantage of the transcranial magnetic stimulation system of our disclosure is the provision of an imaging device or camera central to a center of the TMS coil head configuration, which permits direct visualization of the TMS coil(s) on the patient's head. Conventional or so-called “donut” coils TMS coil head 102A, 102B employed in prior art TMS coil heads as illustrated in FIGS. 10 and 11 are geometrically unsuited for incorporation of an imaging device or camera centrally located between the coils.

In accordance with the present disclosure, we provide TMS coil configuration that permits the positioning of one or more imaging devices, i.e., one or more cameras including a single camera central to the center of the TMS coil head. However, in order to facilitate positioning a camera central to the center of the TMS coil head, we cannot simply wrap or tie our coils together as in the case of prior art traditional “donut” coils 102A, 102B using string 104 or tape 106 to bind the wires together (FIG. 10). Rather, in accordance with the present disclosure we provide the inside of the bottom and/or top of the coil head holder with a plurality of spacers or pegs for holding the wires in place.

Conventional prior art transcranial magnetic stimulation systems also rely on circulating a coolant through the TMS coil head to dissipate excess heat and keep the TMS coil head at a proper and comfortable temperature for the patient. However, providing our novel wire coil geometry that permits us to locate an imaging device including a camera central to the center of the TMS coil head prevents cooling the TMS coil head by continuously circulating a cooling fluid through the coils. Accordingly, in accordance with the present disclosure we employ a phase change material (PCM) permanently packed around the wires in our TMS coil head. The PCM has sufficient thermal energy absorption capacity to cool the coils for the duration of a treatment. Accordingly, in accordance with further aspect of the disclosure, the TMS coil head is configured to be readily swapped out between treatment sessions. To facilitate this, in accordance with the present disclosure, we provide a detachable cable configured to be detachable fixed to the TMS coil head or to the TMS pulse generator. Traditional TMS coils circulate cooling fluid through a cooler so that they can be used non-stop. In accordance with the present disclosure, each TMS system is provided with multiple TMS coils so that the health care provider readily can swap out coil heads between patients. Thus, as distinguished with conventional prior art TMS coil heads that are configured to circulate coolant to the TMS coil heads and also energize the coil(s), our TMS coil head is far simpler in construction and does not need to be configured to circulate a coolant. Also, with our PCM cooled coil heads, the cable needs only a single junction, i.e., to plug into a TMS pulse generator.

In another aspect, with our PCM cooled coil heads, we provide a detachable power cable that can also detach from the TMS coil head, configured to deliver electrical power from a pulse generator to the TMS coil head. This is another advantage over conventional TMS coil heads, which require fixed and expensive cabling to both circulate coolant and deliver electrical pulses.

Employing a PCM as a coolant sealed in the TMS coil head provides additional advantages. For one, the density of PCM is much lighter than traditional liquid coolants such as Galden® HT 135. As a result, our TMS coil head is lighter in weight which means we do not require TMS coil holder arms which are heavy, bulky and difficult to use (see FIG. 11). Additionally, because our TMS coil heads are lighter in weight, we can use light weight compact ergonomically shaped handled on the TMS coil heads on the top of the coil head, as opposed traditional TMS coil heads as illustrated in FIG. 11 that have a “paddle” handle 110 extending out from a side of the coil head 112, which forces the operator to handle the coil head like a sword which is tiring for the operator.

In another aspect, the PCM further facilitates thermal dispersions by including thermally conductive materials such as metal fines, e.g., copper, tin or aluminum, carbon allotropes such as graphite or graphene, or a thermal paste.

Our TMS coil head shape with a compact ergonomically shaped handle on the top of the coil head also permits us to mount the power cable through the top of the coil head. This provides us with another advantage over conventional paddle shaped coil heads in which the power hoses are connected through the handle.

In one aspect, the TMS coil head further comprises a permanent or removable mounting joint on a top of the coil head, vertically centered on a central vertical axis of the coil winding.

In another aspect, a complementary fixture to which the mount joint connects, has a single action quick release mechanism.

More particularly, one aspect of the disclosure provides a TMS coil head configured to be placed over a target brain region for treatment, wherein the TMS coil head comprises a housing containing one or more coil windings within the housing, and a PCM in contact with the one or more windings within the housing.

In one aspect the TMS coil head includes one or more imaging devices including an imaging device located central to a center of the TMS housing and configured to permit direct imaging of the center of the TMS coil housing on a patient's head.

In one aspect the coil windings are located to either side of the center of the TMS coil head,

In one aspect the TMS coils to either side of the center of the TMS coil head are mirror images of one another.

In another aspect the housing contains a base and a top, and wherein the TMS coils are fixed in position around spacers or pegs extending from the base or the top.

In another aspect the TMS coils are fixed in position around spacers or pegs extending from the base, and held down by pegs extending from the top, or vice versa.

In a further aspect the coils are adhesively held in place in the base or the top.

In another aspect, the TMS coil head further comprises an ergonomically shaped handle affixed to a top of the TMS coil head.

In a further aspect the TMS coil head further includes heatsink elements in contact with the PCM.

The present disclosure also provides a TMS system comprising: (1) a pulse generator; and (2) an TMS coil head including a PCM as above described.

In one aspect the TMS system further comprises one or more imaging devices including a single imaging device located central to a center of the TMS coil head and configured to permit direct imaging of the center of the TMS coil head on the patient's head.

In a further aspect the TMS system includes one or more imaging devices comprising one or more cameras, including a single camera central to the center of the TMS coil head.

In another aspect, the TMS system includes one or more imaging devices comprising one or more visible light imaging cameras, one or more ultraviolet light imaging cameras or one or more infrared imaging cameras.

In another aspect, the TMS system one or more imaging devices also comprises two or more cameras located to sides of the TMS coil head.

In a further aspect, the TMS system one or more imaging devices also comprises two or more cameras located away from the center but within a housing of the TMS coil head.

In yet another aspect, the TMS system further comprises one or more accelerometers configured to sense orientation placement or changes in orientation of the TMS coil head.

In a further aspect, the TMS system further comprises one or more contact sensors configured to detect contact and force between the TMS coil head and the patient's head.

In a further aspect, the TMS system one or more contact sensors comprise one or more force-sensitive resistors, one or more capacitive touch sensors or one or more ultrasonic position/touch sensors.

In a further aspect, the TMS system further comprises one or more imaging devices external to the TMS coil head and configured to permit simultaneous visualization of the patient's head as well as the TMS coil head.

In a still further aspect, the TMS system one or more imaging devices external to the one or more TMS coil head comprise one or more cameras, one or more LIDAR detectors, or one or more ultrasonic detectors.

In a further aspect the TMS system further comprises a memory device configured to create a record of the TMS coil head position before and during treatment.

In another aspect, the TMS system one or more imaging devices are configured to transmit an image of the patient's scalp vasculature, the patient's skin patterns, the patient's skull bone structure, or the patient's brain tissue, as a case may be.

The present disclosure also provides a treatment cap configured to provide visual guidance for a medical procedure, comprising a skull cap having a pointed midline brim configured to align to the patient's nasion, and/or indicia markings on both sides of the cap configured to align to the tragus of a patient's ears.

The present disclosure also comprises a TMS kit comprising a TMS system, comprising: i) a pulse generator; ii) a TMS coil head including a PCM as above described, and configured to be placed over a target brain region for treatment; and iii) a patient treatment head cap having a brim that comes to a point on the midline, and having indicia markings configured to overlie target locations on the head of the patient.

In one aspect the TMS kit includes a treatment cap having a pointed midline brim configured to align to the patients nasion, and/or indicia markings including tragus markings on both sides of the cap configured to align with the tragus of the patient's ears, or a point bisecting a line connecting the patient's pupils.

In one aspect the TMS kit further includes a smartphone camera configured to image a position of the head cap.

In a further aspect the neuronavigated TMS kit further comprises one or more imaging devices including a single imaging device central to a center of the TMS coil head and configured to permit direct visualization of the center of the TMS coil head.

In a further aspect the neuronavigated TMS kit one or more imaging devices comprises one or more visible light imaging cameras, one or more ultraviolet light imaging cameras or one or more infrared imaging cameras.

In another aspect the neuronavigated TMS kit further comprises one or more contact sensors configured to detect contact and force between the TMS coil head and the patient's head.

In a further aspect the neuronavigated TMS kit the one or more imaging devices also comprises two or more cameras located to sides of the TMS coil head.

In a further aspect the neuronavigated TMS kit one or more imaging devices also comprises two or more cameras located away from the center but within a housing of the TMS coil head.

In another aspect the neuronavigated TMS kit further comprises one or more accelerometers configured to sense orientation placement or changes in orientation of the TMS coil head.

In another aspect the neuronavigated TMS kit one or more contact sensors comprises one or more force-sensitive resistors, one or more capacitive touch sensors, or one or more ultrasonic position/touch sensors.

In a further aspect the neuronavigated TMS kit further comprises one or more imaging devices external to the TMS coil head and configured to permit simultaneous visualization of the patient's head as well as the TMS coil(s).

In another aspect the neuronavigated TMS kit one or more imaging devices comprises one or more cameras, one or more LIDAR detectors, or one or more ultrasonic detectors.

In a further aspect the TMS kit further comprises a memory device configured to create a record of the TMS coil head position before and during treatment.

In another aspect the neuronavigated TMS kit one or more imaging devices are configured to transmit an image of the patient's scalp vasculature, the patient's skin patterns, the patient's skull bone structure, or the patient's brain tissue, as a case may be.

The present disclosure also provides a method for stimulating a target brain region by TMS, which method comprises: i) providing the neuronavigated TMS system including a TMS coil head containing a PCM; ii) positioning the TMS coil head over the target region using the single imaging device central to the center of the TMS coil head and configured to permit direct visualization and placement of the TMS coil head over the target brain region; iii) activating and deactivating the TMS coil head according to a treatment protocol; and iv) passively cooling the TMS coil head by contact with a contained PCM.

In one aspect the method includes the step of providing the patient with a treatment head cap having indicia markings in the form of at least one of a grid, text and color markings configured to overlie anatomical locations on the head of the patient; and positioning the TMS coil head over the target brain region using one or more imaging device to visualize placement of the TMS coil head relative to the indicia. In a particular embodiment the treatment cap has a pointed midline brim configured to align to the patient's nasion, and/or indicia markings on both sides of the cap configured to align with the tragus of the patient's ears.

In another aspect correct positioning of the TMS coil head is prompted by at least one of visual, auditory, and haptic feedback.

TMS pulses are more comfortable for the patient at specific rotations. For example, the TMS coil rotated to 90 degrees might be more comfortable than 0 degrees. However, one problem delivering stimulation at some rotations of conventional paddle shaped coils heads is that the power cable and cooling hoses coming out of the TMS coil head paddle handle may droop down across the patient's face. Patients find this uncomfortable. So rather than position the coil at an annoying position, our system permits us to rotate the coil at 180 degrees relative to the annoying position, and then reverse the polarity of the electricity. Together this still creates the same electric field. Essentially, a normal waveform in one direction is equivalent to the reversed waveform, with the coil upside down.

This is rotation-induced flip in polarity is a unique possibility of our system because: 1) we have the only camera+cap with indicia that detects coil rotation and thus allow rotation of the coil head, and 2) our power electronics can effortlessly reverse the waveform. We also can cover the case where the operator may manually device to flip the waveform polarity if they are just manually using the device.

### BRIEF DESCRIPTION OF THE DRAWINGS

Further features of the disclosure will be seen from the following detailed description, taken in conjunction with the accompanying drawings, wherein:

FIG. 1 is a schematic view of a conventional frameless stereotaxic MRI-guided neuronavigation system, in accordance with the prior art;

FIG. 2 is a schematic view of a TMS system in accordance with an embodiment of the instant disclosure;

FIG. 3 is a schematic view of a TMS coil in accordance with an embodiment of the instant disclosure;

FIG. 4 is a bottom plan view of a TMS coil element of with an embodiment of present disclosure;

FIG. 5 is a block diagram of a power and control circuit of a first embodiment of the present disclosure;

FIGS. 6A and 6B are perspective views of a cap element of an embodiment of the present disclosure;

FIGS. 7A-7D are perspective views of alternative cap elements of the present disclosure;

FIGS. 8A-8C are views of a patient's skin, vasculature, bone, and cortical elements of the head;

FIG. 9 is a flow diagram of a method of an embodiment of the present disclosure;

FIG. 10 is a top plan view showing coil windings of a conventional prior art TMS coil head;

FIG. 11 is a perspective view of a conventional prior art TMS system with a conventional prior art TMS coil head;

FIG. 12 is a plan view and FIG. 12A a close-up plan view of a partially disassembled TMS coil head in accordance with an embodiment of the present disclosure;

FIG. 13 is a cross sectional view of a TMS coil head in accordance with an embodiment of the present disclosure;

FIG. 14 is a perspective view and FIG. 14A is a close up view of a TMS coil head in accordance with an embodiment of the present disclosure;

FIG. 15 is a cross-sectional view of a detachable power cable in accordance with an embodiment of the present disclosure; and

FIG. 16 is a schematic view of a mapping technique in accordance with the present disclosure.

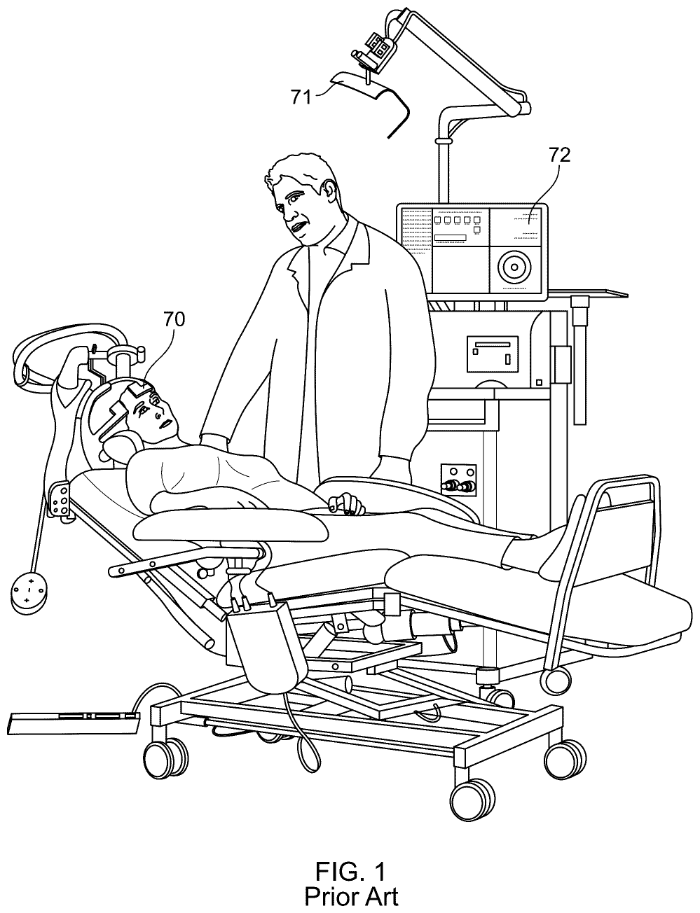

### DETAILED DESCRIPTION OF THE DISCLOSURE

Spatially relative terms, such as “inner,” “outer,” “beneath,” “below,” “lower,” “above,” “upper,” “base,”, “top”, and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly

As used herein the term except as otherwise stated transcranial magnetic stimulation (TMS) coil or coils and TMS coil head shall mean the magnetic induction coils per se and their housing.

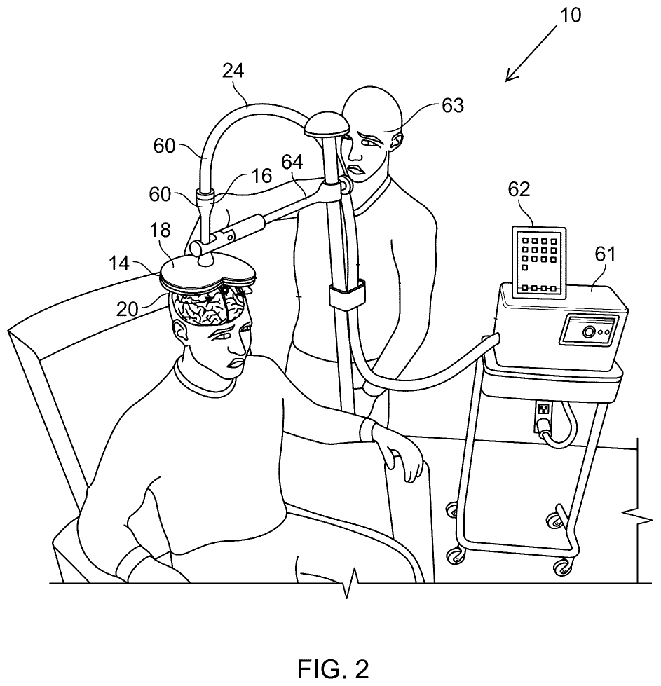

Referring to FIGS. 2-5, a neuronavigated transcranial magnetic stimulation system 10 in one embodiment includes one or more TMS coils, which itself consists of magnetic induction windings 12 in a housing 14 with leads in a cable 24. Alternatively, housing 14 may be connected to an inanimate support mechanism. Housing 14 includes a handle 16 sized for a human hand. Housing 14 includes a top surface 18 and a bottom surface 20, which can be arched to facilitate closer mating with the head of a patient.

The neuronavigated transcranial magnetic stimulation system 10 also includes a pulse generator 61 with an internal control unit and associated power source. The pulse generator sends electricity to windings 12 through a cable 24. The pulse generator 61 may be configured to communicate with smartphone, tablet or PC 62 having a program for the device to send parameters to the pulse generator 61 or for the device to receive data back from the pulse generator. The neuronavigated transcranial magnetic stimulation system 10 is designed to treat and/or ease certain symptoms by applying magnetic stimulation with a certain intensity and frequency through a patient's skull to a target area 26 in the brain within the patient's skull 28. The coil 60 may be held in place by an operator 63, coil-holder 64, or both.

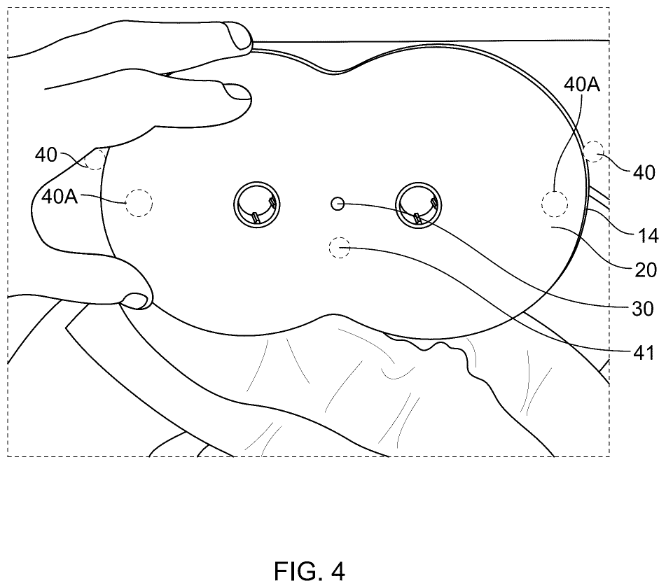

Referring in particular to FIG. 4, housing 14 includes an imaging device 30 configured to face downward from bottom surface 20, i.e., towards the head of a patient when in use, to permit direct visualization of the patient's head. In one embodiment, an imaging device 30 is located centrally relative to the magnetic induction coils. Imaging device 30 preferably comprises a camera which may be a visual light imaging camera, an ultraviolet light imaging camera or an infrared imaging camera.

Alternatively, as shown in phantom at 40 (FIG. 4), the imaging devices may include spaced imaging devices located away from the center, at the sides of the magnetic induction coil's windings 12. Alternatively, two or more imaging devices shown in phantom at 40A, may be placed facing downward, away from the center of the housing, but within the housing spaced from one another at the same distance from the center of the windings 12, or attached adjacent to the edges of the windings 12.

Also, if desired, one or more contact sensors 41 configured to detect force between the coils and the patient's head may be provided, carried on the underside of housing 14. The contact sensors 41 may comprise one or more force-sensitive sensors, one or more capacitive sensors, or one or more infrared sensors.

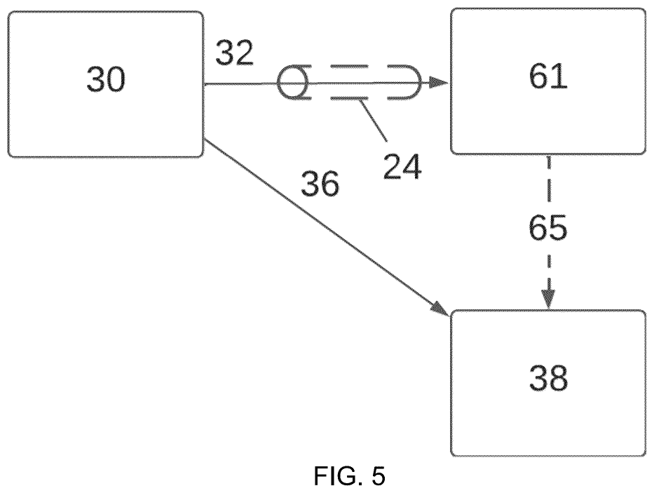

Referring in particular to FIG. 5, in one embodiment, imaging device 30 is connected via cable 36 to a display and memory device 38 which may be a smartphone, tablet or PC. In another embodiment, imaging device 30 is connected via a cable 32 to the pulse generator 61. Cable 32 may travel through cable 24. Optionally, pulse generator 61 may pass imaging information to device 62 which may be a smartphone, tablet, or PC. This connection 65 may be wired, or wireless via Bluetooth, Wi-Fi, NFC or the like.

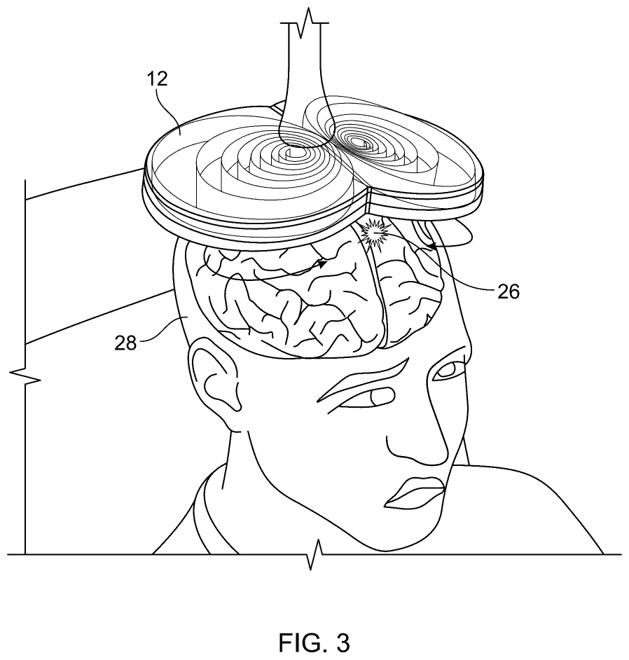

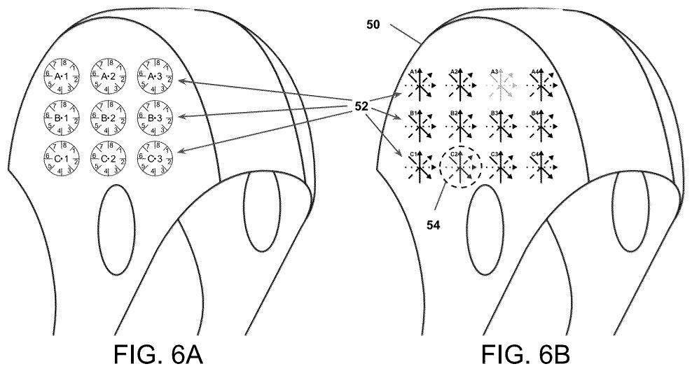

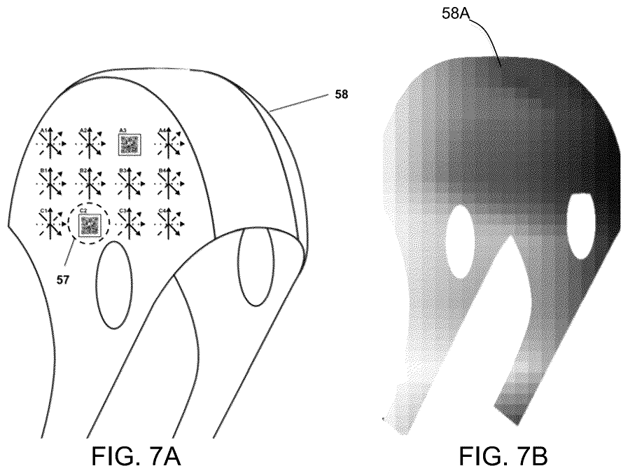

Referring also to FIGS. 6A and 6B, in a preferred embodiment of the disclosure, we provide a treatment cap 50 sized and shaped to fit snugly over a patient's head. The cap may be composed of material intentionally designed to stretch, to accommodate a defined range of head sizes slightly larger than its unstretched size. Preferably cap 50 may be provided in a kit with several different sizes to fit different size patients. Typically, five sizes are sufficient to fit the majority of adult heads, a sixth size for youths, and a seventh size for small children and infants. Cap 50 includes indicia 52 in the form of a specific grid with anatomical markers printed on the cap. The indicia or markers may include text, color and/or symbols to identify specific target locations in the head of the wearer and/or shapes and patterns to indicate orientation for the TMS windings 12 (FIG. 3) to facilitate the magnetic induction in the correct location and orientation. These indicia may also be comprised of symbols, QR codes, color spectra, or any combination thereof. The indicia may be identical across numerous copies of the cap produced. Alternatively, a cap 50 may instead have unique indicia 57 in one or more locations such that the cap and any location on it can be uniquely distinguished from any other (FIG. 7A). Alternatively, cap 58A may have printed patterns or color gradations to guide placement (FIG. 7B). The caps also may include indicia to personalize a cap to an individual patient.

As mentioned supra, the current standard of care is to place the treatment cap just above the eyebrows, measure the distance from nasion to cap brim, and then for every subsequent session, try and place the cap back exactly that same distance, remeasuring each time. This is laborious and error prone.

Referring to FIGS. 7C and 7D, in accordance with another embodiment the present disclosure rather than use a cap with a flat brim 300, the present disclosure uses unique cap geometry where the brim 300 comes to a point 302 on the midline. This point can be immediately visually confirmed to be correctly placed or not without the need for measuring tape.

Additionally, we may place indicia 304 on the cap that denotes where the cap should be with respect to the tragus 306 (the point flap of skin on the ear), as an additional marker to ensure reliable fit of the cap on the head.

With both the pointed brim 300, and tragus markers 306 on both sides, these three markers may be used not only for basic visual confirmation, but an AI algorithm optionally can be implemented to ensure proper cap positioning, using a smartphone 310 camera and slowly wave it around the patient from left to front to right side to ensure the cap is properly positioned.

Yet another feature and advantage of the instant disclosure that results from the provision of a camera central to the center of the TMS coils and a cap with indicia as above described is that we can detect coil rotation, and if desired flip or reverse the waveform polarity by changing, i.e., reversing the polarity of the power electronics to improve patient comfort.

Because the magnetic induction coil's field has a particular orientation (it is directional, not symmetric), the angle at which the magnetic induction coil is placed over a given location makes a meaningful difference in how patients experience the procedure. Specifically, even over the exact same central location, positioning the coil at different angles will activate different central and peripheral nerves. In the latter case, this may cause uncomfortable sensations at some angles, but not others. For example, at some angles, a patient's jaw may jitter during TMS, while not at others. Thus, the indicia's shape and pattern uniquely identifies each angle at which the magnetic induction coil may be placed so that, in conjunction with the camera, a viewer can see if they are properly and consistently aligned. Notably, the indicia are neither radially, nor bilaterally symmetric, and thus a rotation of 180 degrees of the magnetic induction coil will result in a different perspective on any given marker so that it is again, uniquely identified. Similarly, the text and color combination of each anatomical marking uniquely identifies the location. Locations commonly used as stimulation targets or reference locations in the therapeutic TMS community are further differentiated using color, to allow for quick and robust setup. This permits the healthcare provider to ensure that the magnetic induction windings 12 are properly positioned on the head of the wearer, and not skewed or tilted. We also can infer the coil's distance from the head of the wearer due to image size, to ensure that the coil is in full contact when seen by the imaging device.

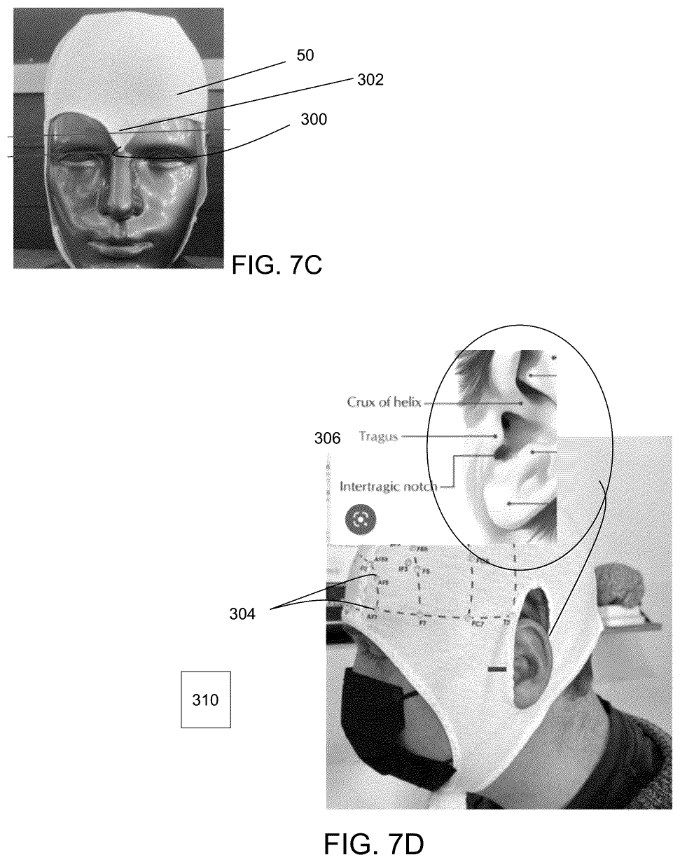

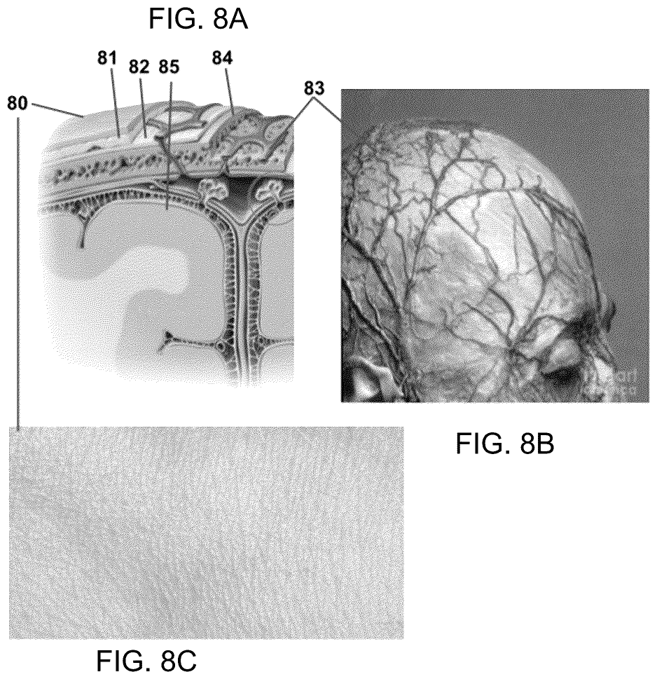

In another embodiment no specialized treatment cap is employed. Instead, patient-specific anatomical features are used to locate and maintain the coil in position. Referring to FIGS. 8A-8C, these features may comprise the epidermis 81, dermis 82 and hypodermis 82 patterns, scalp vascularization 83, bone density 84 and neural tissue configuration 85 obtained by an optical or infrared camera and/or through functional near infrared spectroscopy.

This makes it possible to perform a multi-modal “fingerprint” of the precise location of the stimulation target and determine the location of the magnetic induction coil accordingly based on these individually unique anatomical features of each patient's scalp itself rather than the premarked cap. The image patterns may be recorded and saved for future treatments.

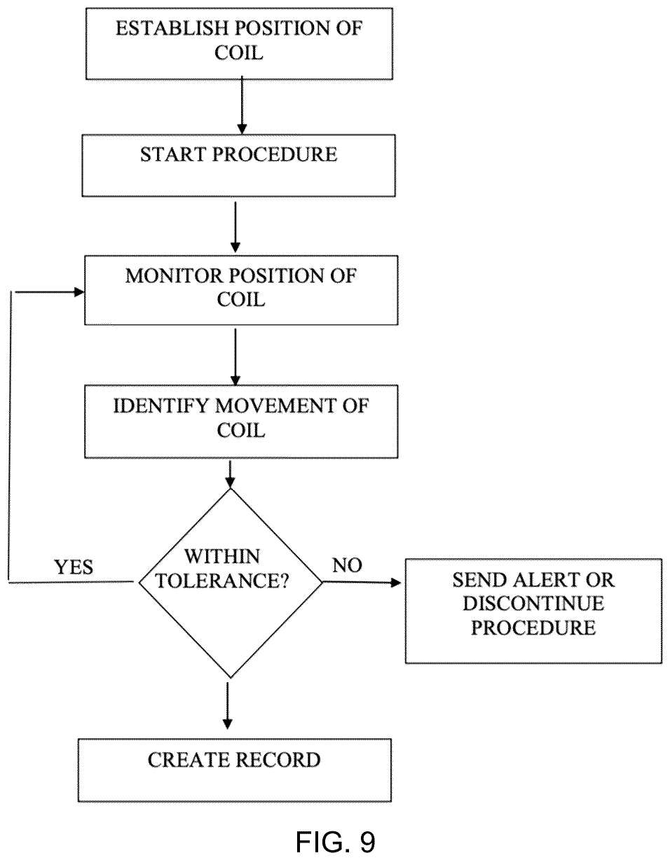

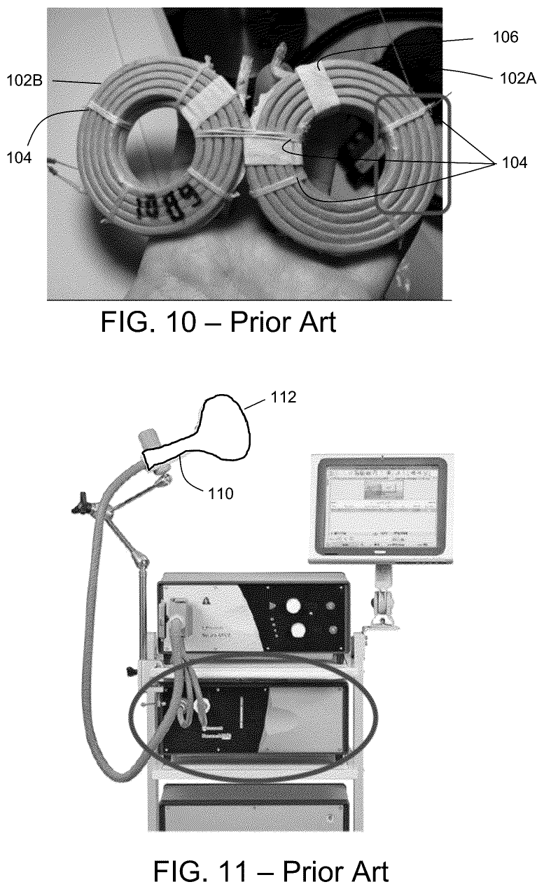

A feature and advantage of the present disclosure derives from use of one or more imaging devices internal to the TMS coil housing 14 which not only ensures proper placement of the transcranial magnetic stimulation system, but also permits continuous monitoring of placement and also includes an ability to record and/or transmit placement data in real time during the entire procedure. Also, by providing target indicia 54 on the cap, the healthcare provider can accurately locate the transcranial magnetic stimulator over a target area of the brain. Alignment can be prompted via visual, auditory and/or haptic feedback. Referring to FIG. 9, another feature and advantage of the present disclosure that results from the use of built-in imaging devices is that, should the coil be dislodged or moved (due to movement by the patient for example), an alert signal can be generated for the health care provider. Moreover, to protect the patient from possible harm, the magnetic stimulator can be programmed to not start until the magnetic induction coil is properly positioned, and to turn off or pause delivery of stimulation pulses when misalignment of the magnetic induction coil exceeds a certain tolerance, prompting correction of the position by the operator before proceeding.

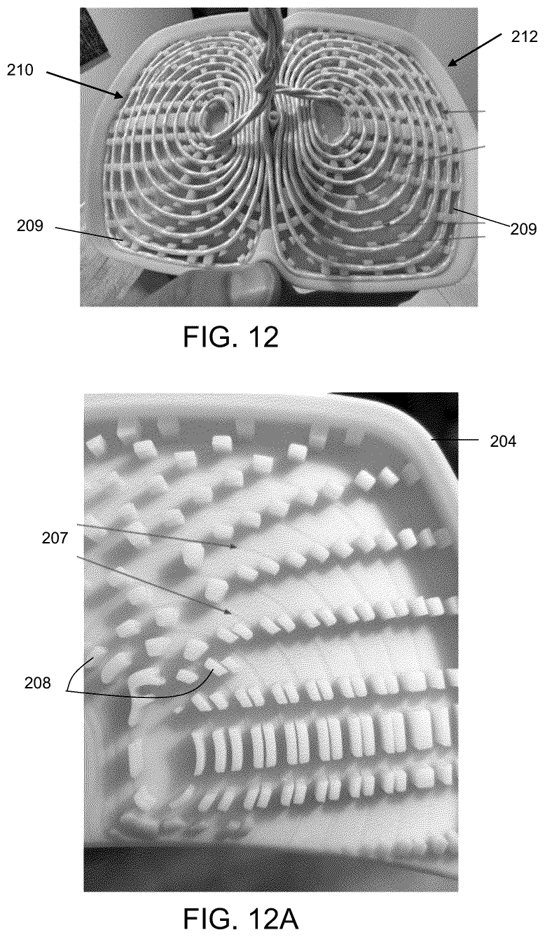

Referring to FIGS. 12, 12A, 13 and 14 a TMS coil head 200 in accordance with another embodiment of the present disclosure comprises a fluid tight housing 202 having a base 204 and a top 206. Housing 202 has a slightly concave shape for approximating the head of a patient. Base 204 includes a plurality of inlaid winding paths 207 and spacers or pegs 208 for guiding placement of the wiring within the housing. The wiring comprises the treatment coil for providing TMS magnetic stimulation to the patient's brain. The wiring includes a wiring structure comprising a continuous wire 209 wound in continuous series of wire loops 210 and 212. Wire loops 210 and 212 are essentially mirror images of one another. Wire 209 includes a conductive coil and a dielectric coating.

Wire loops 210, 212 typically are glued in position by an adhesive laid in channels 207 and/or between pegs 208, before the wire loops 210, 212 are placed into position in the base 204. Additional pegs located on and extending downwardly from the top 206 may be provided for pressing down on and holding the wire loops 210, 212 in position. Alternatively, the channels and pegs may be formed in/on the inside of the top 206, and the wire loops placed into position in the top 206. The pegs also ensure that the wire loops are spaced from one another to permit a phase change material to flow between the loops and contact the wires to increase thermal contact with the wires. The wire loops 210, 212 may be placed into position by hand or by robot. The free ends of the wire 209 is then threaded through a hole or fitting (not shown), in the top 206, and subsequently connected to a power cable which in turn is connected to a power generator of a neuronavigated transcranial magnetic stimulation system, e.g., as above described. However, the conventional circulating coil head cooling system may be bypassed since it is not needed as discussed below.

A PCM such as PulseICE Organic A36 that is normally solid at ambient temperature, is then heated to melting, and the melted PCM is then poured into the bottom or top of the housing as the case may be to cover and encase the wire loops 210, 212. The PCM then is allowed to cool and solidify. PCMs have an advantage over static fluids in that they absorb much more thermal energy during the act of melting. By way of example Galden®HT 135 which traditionally has been used as a circulating heat transfer agent with conventional TMS coil heads absorbs 0.23 J/gK, whereas PulseICE Organic A36 PCM absorbs about 250 J/g just by melting. However, unlike static heat transfer agents such as Galden® HT 135, PCM's are poor conductors of heat over distance. Thus, PCM's only work when they are in close contact with a heat source. By laying the wire coils with spacing between the loops, and by solidifying the liquid PCM in situ in contact with the wire coils, in accordance with the present disclosure, we maximize thermal heat transfer from the wire coils to the PCM.

As noted supra, a preferred PCM material is PulseICE Organic A36. However, we also may mix other materials with the PCM to improve the PCM thermal conductivity, such as metal fines, e.g., of copper, tin or aluminum, carbon allotropes, e.g., graphite, or graphene, or a thermal paste. We also can affix solid heat sinks that are not very electrically conductive, e.g, aluminum oxide, to the wire coils.

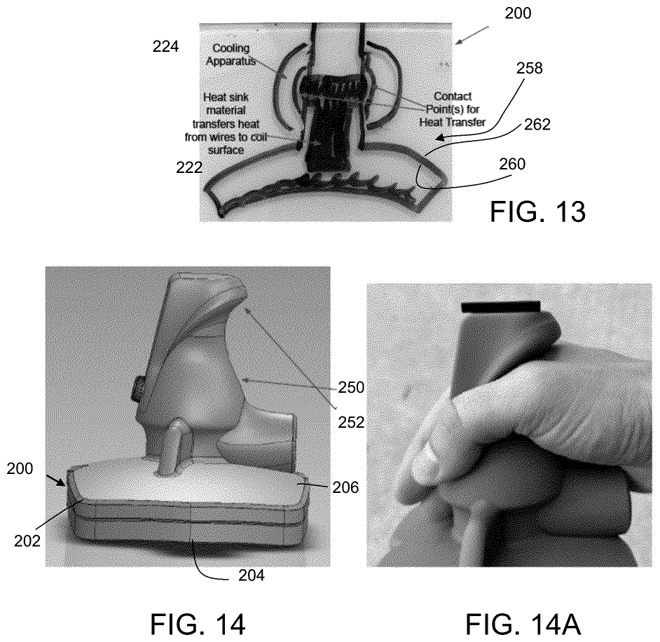

Once the PCM is solidified, the coil head is assembled, base 204 and top 206 are sealed together, a power cable 220 is attached, and the coil head 200 is ready to use. The PCM in the coil head has sufficient cooling capacity to last the length of a typical treatment, i.e., 0.5 to 10 minutes. Once treatment is complete, the coil head is allowed to cool, whereupon the PCM solidifies and is ready for reuse. Referring to FIG. 13, to speed cooling between uses, the coil head 200 may include solid heat-sink elements 222 extending from the coils to the surface of the coil head 200. The coil head 200 can then be placed onto a cooling apparatus 224. In one embodiment, the cooling apparatus 224 is passive and ambient air is used to cool the heat-sink elements which then transfer cooling into the PCM inside the coil head 200. In another embodiment, the cooling rate is increased by an active cooling device such as Peltier Thermocouple.

Also in order to reduce or eliminate unwanted electromagnetic interference emissions from the TMS pulse generator or the TMS coil head, rather than form the TMS pulse generator or the TMS coil head enclosure of heavy metal, we can form the TMS pulse generator or the TMS head enclosure 258 of a light weight polymeric material, and coat the inner and/or outer surfaces 260, 262 of the enclosure 258 with electrically conductive materials, such as silver, graphene, copper, carbon nanotubes and mixtures thereof.

Referring also to FIGS. 14 and 14A, because the density of the PCM's we use is so much lighter than the density of traditional coolants such as Galden®HT 135, our coil head 200 is relatively light weight and easily moved by hand. To facilitate holding and moving our coil head 200, we provide our coil head 200 with a handle 250 having an overhang 252 that is ergonomically sized and shaped for the human hand. The handle also permits the operator to “free hand” the coil head 200, facilitating movement of the coil head 200 over the patient's head. The handle is both easy on the hand of the operator, but also sufficiently spaced from the wires within the coil head 200 so that the TMS pulses don't shock the operator's hand. Various changes may be made in the foregoing disclosure. For example, the treatment cap also may be advantageously used for directing radiation for radiation oncology treatment of the brain.

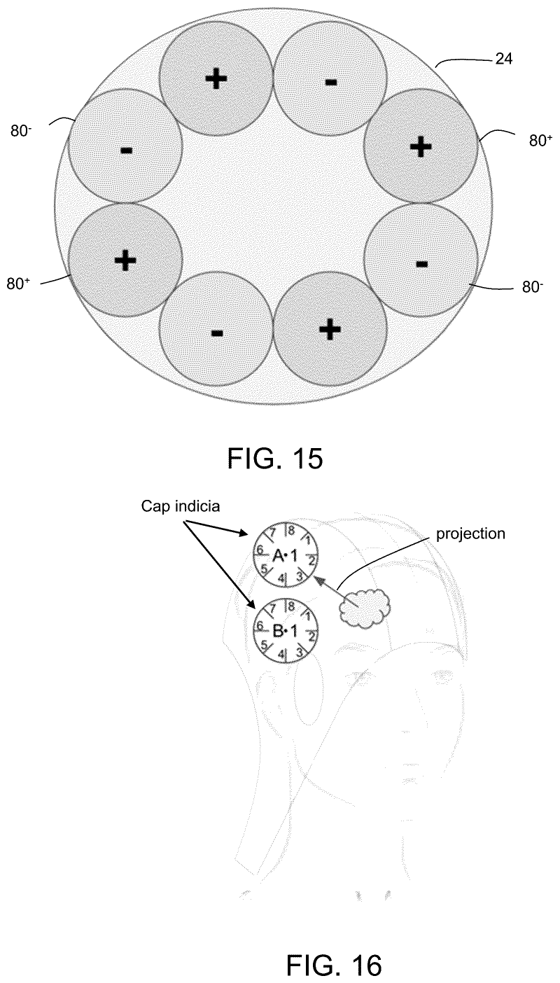

For example, referring to FIG. 15, the power cable 24, which may be detachable from the TMS head and/or the pulse generation, may comprise a plurality of conductors 84⁺, 80⁻, wherein half the conductors, i.e., the conductors marked with a plus “+” sign represent flowing into the TMS coil, while the conductors marked “−” represent current flowing out of the TMS coil head.

Also, in yet another aspect of the disclosure illustrated in FIG. 16, we choose what indicia on our head cap is the best location to treat a patient as follows: we give patients an MRI or fMRI in order to determine anatomically or functionally, what are the exact coordinates of the brain region to treat. However, TMS is non-invasive so we can't actually make target location within a patient's head. Accordingly, we take those coordinates (either in the head in 3d, or on the scalp in 2d) and project or map those location to indicia on our cap, and optionally with a particular rotation specified.

Still other changes may be made without departing from the spirit and scope thereof.

What is claimed:

1. A transcranial magnetic stimulation (TMS) kit comprising a transcranial magnetic stimulation system, comprising:
i) a pulse generator;
ii) a TMS coil head as claimed in claim 1, configured to be placed over a target brain region for treatment wherein the TMS coil head comprises a housing containing one or more coil windings within the housing, and a phase change material (PCM) in contact with the one or more windings within the house; and
iii) a patient head cap having a brim that comes to a point on the midline, and having indicia markings configured to overlie target locations on the head of the patient.

2. The TMS kit of claim 1, including indicia markings including tragus markings on both sides of the cap.

3. The TMS kit of claim 1, further including a smartphone camera configured to image a position of the head cap.

4. The TMS kit of claim 1, further comprising one or more imaging devices including a single imaging device central to a center of the TMS coil head and configured to permit direct visualization of the center of the TMS coil head.

5. The TMS kit of claim 4, wherein the one or more imaging devices comprises one or more visible light imaging cameras, one or more ultraviolet light imaging cameras or one or more infrared imaging cameras.

6. The TMS kit of claim 1, further comprising one or more contact sensors configured to detect contact and force between the TMS coil head and the patient's head.

7. The TMS kit of claim 4, wherein the one or more imaging devices also comprises two or more cameras located to sides of the TMS coil head.

8. The TMS kit of claim 4, wherein the one or more imaging devices also comprises two or more cameras located away from the center but within a housing of the TMS coil head.

9. The TMS kit of claim 1, further comprising one or more accelerometers configured to sense orientation placement or changes in orientation of the TMS coil head.

10. The TMS kit of claim 6, wherein the one or more contact sensors comprises one or more force-sensitive resistors, one or more capacitive touch sensors, or one or more ultrasonic position/touch sensors.

11. The TMS kit of claim 4, further comprising one or more imaging devices external to the TMS coil head, and configured to permit simultaneous visualization of the patient's head as well as the TMS coil head.

12. The TMS kit of claim 4, wherein the one or more imaging devices comprises one or more cameras, one or more LIDAR detectors, or one or more ultrasonic detectors.

13. The TMS kit of claim 1, further comprising a memory device configured to create a record of the TMS coil head position before and during treatment.

14. The TMS kit of claim 4, wherein the one or more imaging devices are configured to transmit an image of the patient's scalp vasculature, the patient's skin patterns, the patient's skull bone structure, or the patient's brain tissue, as a case may be.

15. A method for stimulating a target brain region by transcranial magnetic stimulation (TMS), which method comprises:
i) providing the neuronavigated transcranial magnetic stimulation system as claimed in claim 1;
ii) positioning the TMS coil head over the target region using the single imaging device central to the center of the TMS coil head and configured to permit direct visualization and placement of the TMS coil head over the target brain region; and
iii) activating and deactivating the TMS coil head according to a treatment protocol;
iv) passively cooling the TMS coil head by contact with a contained phase change material (PCM).

16. The method of claim 15, including the steps of providing the patient with a head cap having indicia markings in the form of at least one of a grid, text and color markings configured to overlie anatomical locations on the head of the patient; and
positioning the TMS coil head over the target brain region using one or more imaging devices to visualize placement of the TMS coil head relative to the indicia.

17. The method of claim 15, wherein correct positioning of the TMS coil head is prompted by at least one of visual, auditory, and haptic feedback.

18. A treatment cap configured to provide visual guidance for a medical procedure, comprising a skull cap having a pointed midline brim configured to align to the patient's nasion or a point bisecting a line connecting the patient's pupils, and/or indicia markings on both sides of the cap configured to align to the tragus of a patient's ear.

19. The TMS kit of claim 1, wherein the PCM further includes thermally conductive materials selected from the group consisting of metal fines, preferably copper, tin or aluminum, carbon allotropes, preferably graphite or graphene, or a thermal paste.

20. The TMS kit of claim 1, further comprising a power cable that that is detachable from the TMS coil head and/or from the pulse generator, configured to deliver electrical power from the pulse generator to the TMS coil head.

21. The TMS kit of claim 20, wherein the power cable includes a plurality of conductors wherein half the plurality of conductors carrying current towards the TMS coil head are adjacent to the other half of the plurality of conductors wires carrying current away from the TMS coil head.

22. A transcranial magnetic stimulation (TMS) pulse generator or TMS coil head configured to be placed over a target brain for treatment, having an enclosure formed of a polymeric material, and coated inside and/or outside, at least in part, with an electrically conductive material.

23. The TMS coil head of claim 22, wherein the electrically conductive material is selected from the group consisting of silver, graphene, copper, carbon nanotubes, and mixtures thereof.

24. The method of claim 15, including the steps of providing the patient with a head cap having markings in the form of at least one of a grid, text and color markings configured to overlie anatomical locations on the head of the patient, and positioning the TMS coil head over their target brain region using a projection of coordinates of a target brain region which most closely aligns with a nearest indicia location.
